(19) United States
(12) Patent Application Publication
(10) Pub. No.: US 2021/0267464 A1
(43) Pub. Date: Sep. 2, 2021

(54) BIOSENSOR

(71) Applicant: KYOCERA Corporation, Kyoto (JP)

(72) Inventors: Asao HIRANO, Shinagawa-ku, Tokyo (JP); Takeshi HIGUCHI, Yokohama-shi, Kanagawa (JP)

(73) Assignee: KYOCERA Corporation, Kyoto (JP)

(21) Appl. No.: 17/290,871

(22) PCT Filed: Nov. 6, 2019
(86) PCT No.: PCT/JP2019/043424

§ 371 Date: May 3, 2021

(30) Foreign Application Priority Data
Nov. 15, 2018 (JP) 2018-214807

(51) Int. Cl. A61B 5/0205; A61B 5/1455; A61B 5/026; A61B 5/00

(52) U.S. Cl. CPC A61B 5/0205; A61B 5/14551; A61B 5/0002; A61B 5/6817; A61B 5/741; A61B 5/0261

(57) ABSTRACT

Provided is a biosensor including a main body and a measurement unit. The main body is configured to sandwich a helix of a subject by a first wearing portion and a second wearing portion. The measurement unit measures at least one of percutaneous oxygen saturation (SpO₂) and blood flow amount of the subject.

### CROSS-REFERENCE TO RELATED APPLICATION

This application claims priority to and benefit of Japanese Patent Application No. 2018-214807 filed on Nov. 15, 2018, the entire contents of which are incorporated herein by reference.

### TECHNICAL FIELD

This disclosure relates to biosensors.

### BACKGROUND

A known measurement apparatus is attached to a human body to measure biological information. For example, Patent Literature 1 (PTL 1) discloses an ear-worn apparatus that is worn on an ear to detect biological information and calculates the blood flow amount state value on the basis of the detected biological information.

### CITATION LIST

### Patent Literature

PTL 1: JP2005-192581A

### SUMMARY

### Solution to Problem

A biosensor according to an embodiment includes a main body and a measurement unit. The main body is configured to sandwich a helix of a subject between a first wearing portion and a second wearing portion.

The measurement unit measures at least one of percutaneous oxygen saturation (SpO₂) and blood flow amount of the subject.

### BRIEF DESCRIPTION OF THE DRAWINGS

In the accompanying drawings:

FIG. 1 is a perspective view illustrating an appearance of a biosensor according to an embodiment;

FIG. 2 is a top view of the appearance of the biosensor according to an embodiment;

FIG. 3 is a side view of the appearance of the biosensor according to an embodiment;

FIG. 4 is a side view of the appearance of the biosensor according to an embodiment;

FIG. 5 is a diagram illustrating an example in which the biosensor according to an embodiment is worn on a subject;

FIG. 6 is a diagram illustrating an ear of a subject;

FIG. 7 is a diagram illustrating a function of the biosensor according to an embodiment;

FIG. 8 is a diagram illustrating a function of a biosensor according to a variation of an embodiment;

FIG. 9 is a diagram schematically illustrating an internal structure of the biosensor according to an embodiment;

FIG. 10 is a diagram schematically illustrating an internal structure of the biosensor according to a variation of an embodiment;

FIG. 11 is a diagram schematically illustrating an internal structure of the biosensor according to another embodiment;

FIG. 12 is a diagram schematically illustrating an internal structure of the biosensor according to a variation of another embodiment;

FIG. 13 is a functional block diagram illustrating a schematic configuration of a biosensor and a measurement apparatus according to an embodiment;

FIG. 14 is a flowchart illustrating an example of process executed by the biosensor according to an embodiment; and

FIG. 15 is a functional block diagram illustrating a schematic configuration of the biosensor according to another embodiment.

### DETAILED DESCRIPTION

When measuring the biological information of a subject, if the biological information can be measured stably while reducing physical and mental load on the subject, the convenience of the measuring instrument can be improved. The purpose of this disclosure is to provide a biosensor that can improve the convenience. According to this disclosure, a biosensor that can improve the convenience can be provided. A biosensor according to an embodiment will be described below with reference to drawings.

A biosensor according to an embodiment is worn on an ear of a subject when the biological information of the subject is measured. The biosensor according to an embodiment measures the biological information of the subject while being worn on an ear of the subject. Here, the biological information is any information on a living body, and may include, for example, oxygen saturation, percutaneous oxygen saturation (SpO₂), body temperature, pulse rate, respiration rate, Perfusion Index (PI) value, blood flow amount, blood pressure, and the like. Further, the biological information may include, for example, a relax degree that indicates a physical and mental relax degree of a living body. The biosensor 1 may estimate the state of the subject on the basis of the measured biological information. The state of the subject is any state that occurs in a living body of the subject, and includes a possibility of developing an altitude sickness.

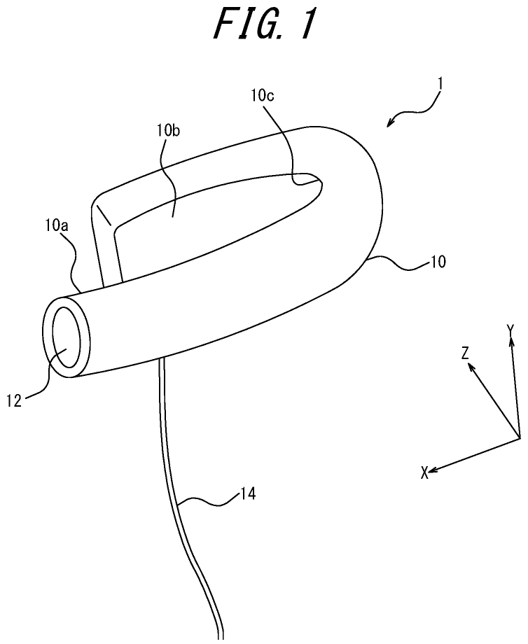

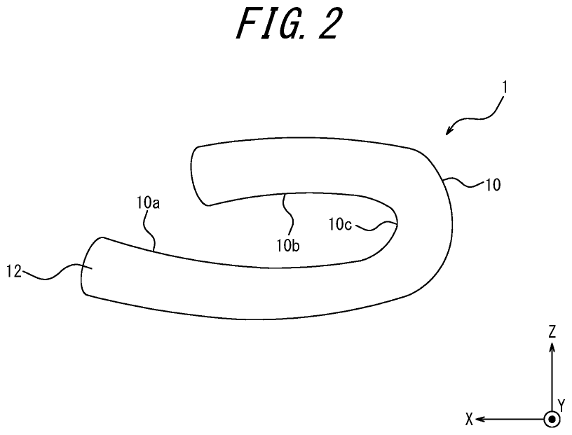

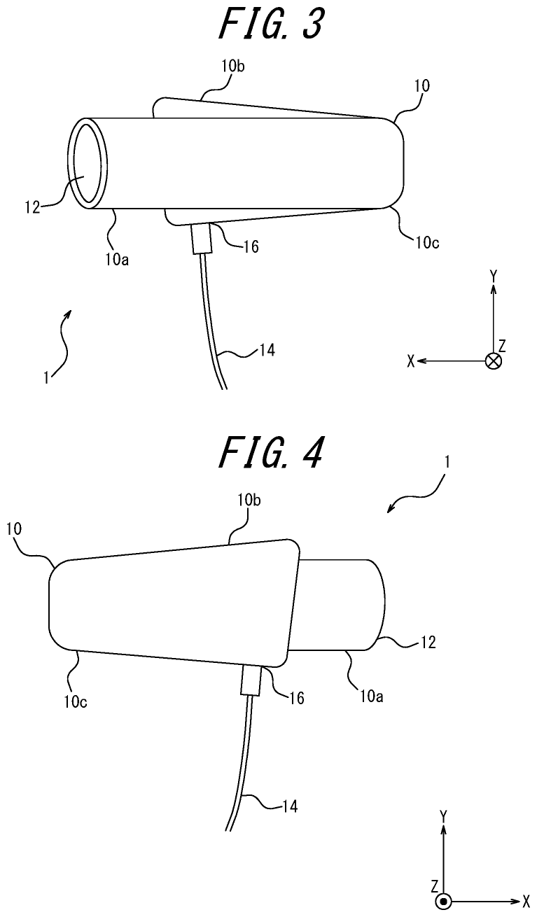

FIG. 1 is a perspective view illustrating an appearance of a biosensor according to an embodiment. FIG. 2 is a diagram of the appearance of the biosensor illustrated in FIG. 1 viewed from above. That is, FIG. 2 is a diagram illustrating a state of the biosensor illustrated in FIG. 1 viewed in the negative direction of the Y-axis. FIGS. 3 and 4 are diagrams of the appearance of the biosensor illustrated in FIG. 1 viewed from side. That is, FIG. 3 is a diagram illustrating a state of the biosensor illustrated in FIG. 1 viewed in the positive direction of the Z-axis. Further, FIG. 4 is a diagram illustrating a state of the biosensor illustrated in FIG. 1 viewed in the negative direction of the Z-axis. In FIGS. 1-4, the positive direction of the Y-axis is also referred to as “up” direction as appropriate.

As illustrated in FIGS. 1-4, the biosensor 1 according to an embodiment includes a main body 10. As illustrated in FIGS. 1-4, the main body 10 includes a first wearing portion 10a, a second wearing portion 10b and a connecting portion 10c.

The first wearing portion 10a is an elongated portion extending substantially parallel to the X-axis direction illustrated in the figure. The second wearing portion 10b is an elongated portion extending substantially parallel to the X-axis direction illustrated in the figure. In FIGS. 1-4, although the first wearing portion 10a is longer than the second wearing portion 10b, these lengths may be changed as appropriate. Further, as illustrated in FIGS. 1-4, the connecting portion 10c connects the first wearing portion 10a and the second wearing portion 10b.

In the main body 10, the first wearing portion 10a, the second wearing portion 10b and the connecting portion 10c may be integrally formed. On the other hand, in the main body 10, at least one of the first wearing portion 10a, the second wearing portion 10b and the connecting portion 10c may be formed separately from the other members. When at least any one of the first wearing portion 10a, the second wearing portion 10b and the connecting portion 10c is formed separately from the other member, they may be attached with appropriate materials such as adhesive.

As described later, the main body 10 of the biosensor 1 is worn on the ear of the subject when the biological information of the subject is measured. In that case, at least one of the first wearing portion 10a, the second wearing portion 10b and the connecting portion 10c may be configured by a flexible material so that physical and mental load on the subject is reduced in a state in which the main body 10 of the biosensor 1 is worn on the ear of the subject. For example, at least one of the first wearing portion 10a, the second wearing portion 10b and the connecting portion 10c may be made of a soft material such as silicone rubber or urethane. On the other hand, at least one of the first wearing portion 10a, the second wearing portion 10b and the connecting portion 10c may have a core part made of a hard material such as plastic or metal and a surface made of a soft material such as silicone rubber or urethane. The main body 10 of the biosensor 1 may be made of various materials so as not to increase the physical and mental load on the subject more than necessary when it is worn on the ear of the subject.

As illustrated in FIGS. 1-4, a sound output hole 12, which is a hole through which sound is output, may be formed at an end opposite to the side connected to the connecting portion 10c of the first wearing portion 10a. As described later, the first wearing portion 10a may have a build-in sound output interface. The first wearing portion 10a can output sound or voice mainly in the positive direction of the X-axis illustrated in the figure by incorporating the sound output interface.

As illustrated in FIGS. 1, 3 and 4, the biosensor 1 may have a cable 14. In this case, the cable 14 may connect the biosensor 1 to an external device such as a measurement apparatus 100 described later. Further, the cable 14 may be configured to be attached to and detached from the main body of the biosensor 1. In FIG. 2, the cable 14 is not illustrated. Further, as illustrated in FIGS. 3 and 4, the second wearing portion 10b of the main body 10 may have a cable connecting unit 16. In this case, the cable 14 may be connected to the cable connecting unit 16. Further, also in this case, the cable 14 may be configured to be attached to and detached from the cable connecting unit 16. FIGS. 1, 3 and 4 illustrate an example in which the cable 14 is connected to the second wearing portion 10b. However, the cable 14 may be connected to any portion of the main body 10 such as the first wearing portion 10a or the connecting portion 10c, for example.

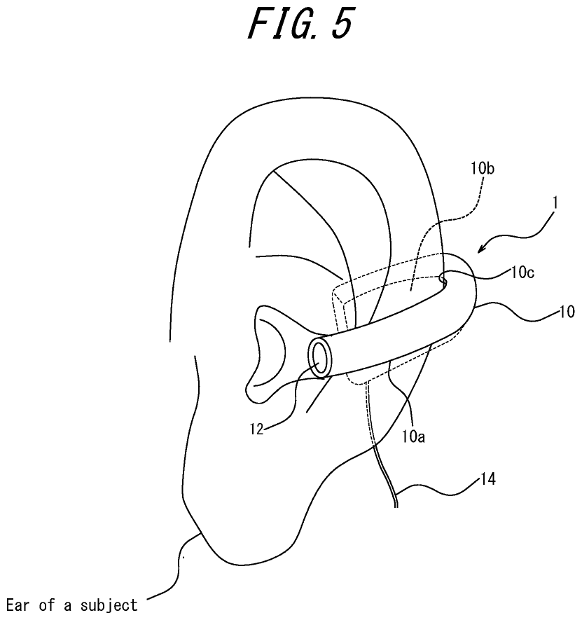

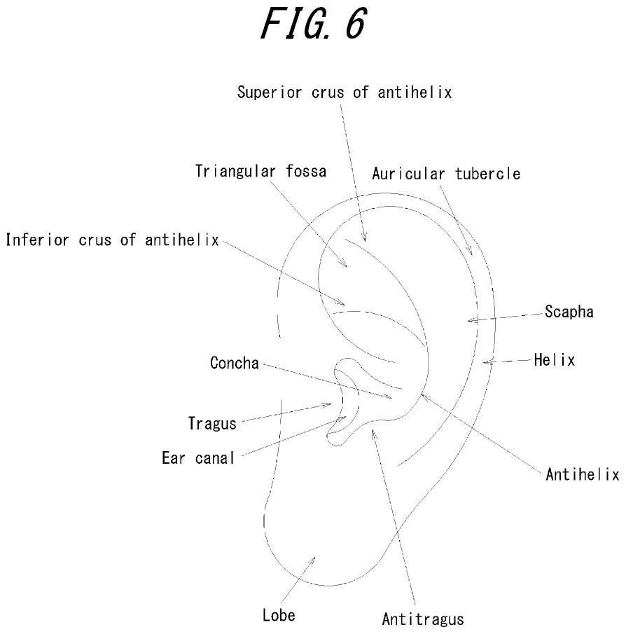

FIG. 5 illustrates a state in which the biosensor 1 is worn on the ear (left ear) of the subject. Further, for your reference, FIG. 6 illustrates part names of the general human ear (left ear).

As illustrated in FIG. 5, the biosensor 1 can be worn on the ear of the subject. In this case, the first wearing portion 10a of the main body 10 may be worn on the front side of the outer ear (auricle), that is, the side of the outer ear having an external auditory canal. Further, the second wearing portion 10b of the main body 10 may be worn on the back side of the outer ear (auricle), that is, the side of the outer ear having no external auditory canal. In more detail, when the main body 10 of the biosensor 1 is worn on the ear of the subject, the helix of the subject may be sandwiched between the first wearing portion 10a and the second wearing portion 10b. In this manner, since the helix of the subject is sandwiched between the first wearing portion 10a and the second wearing portion 10b with an appropriate pressure, the main body 10 of the biosensor 1 is stably maintained at the position of the helix of the subject.

FIG. 5 illustrates an example in which the biosensor 1 for the left ear is worn on the left ear of the subject. However, in an embodiment, the biosensor 1 may be configured for the right ear, and the biosensor 1 for the right ear may be worn on the right ear of the subject. In this case, the biosensor 1 configured for the right ear and the biosensor 1 configured for the left ear may be symmetrical (symmetrical in the Z-axis direction illustrated in FIGS. 1-4).

In this manner, in the biosensor 1 according to an embodiment, the main body 10 is configured to sandwich the helix of the subject between the first wearing portion 10a and the second wearing portion 10b. Thus, according to the biosensor 1 of an embodiment, when the biological information of the subject is measured, the biological information can be stably measured while the physical and mental load on the subject is reduced. Therefore, according to the biosensor of an embodiment, the convenience can be improved.

In FIGS. 1-5, the first wearing portion 10a and the second wearing portion 10b are illustrated as members extending substantially linear in the X-axis direction. However, at least one of the first wearing portion 10a and the second wearing portion 10b may be an appropriately curved member. For example, the first wearing portion 10a may be curved along the depression of the navicular fossa when it is worn on the ear of the subject. Further, the first wearing portion 10a may be curved along the shape of protrusion of the anthelix when it is worn on the ear of the subject. Moreover, the first wearing portion 10a may be curved along the shape of the depression of the concha auriculae when it is worn on the ear of the subject. Further, the second wearing portion 10b may also be curved along the shape of the back side of the outer ear (auricle) when it is worn on the ear of the subject. Moreover, the main body 10 may be configured such that at least one of the first wearing portion 10a and the second wearing portion 10b has plasticity. In this case, at least one of the first wearing portion 10a and the second wearing portion 10b can be deformed along the shape of the ear of the subject.

Further, as illustrated in FIG. 5, the end (sound output hole 12) of the first wearing portion 10a may be located in front of the entry of the external auditory canal of the subject when the main body 10 of the biosensor 1 is worn on the ear of the subject. That is, the first wearing portion 10a may be configured such that the sound output hole 12 will not be inserted into the external auditory canal of the subject when the biosensor 1 is worn on the ear of the subject. In this case, the external auditory canal of the subject is not closed by the sound output hole 12. In this manner, the subject can hear the sound from the surrounding environment while listening to the sound or voice output from the sound output hole 12. Therefore, the subject can recognize the circumstance to significant degree even while measuring the biological information by using the biosensor 1.

On the other hand, as a variation of the embodiment illustrated in FIG. 5, when the main body 10 of the biosensor 1 is worn on the ear of the subject, the end (sound output hole 12) of the first wearing portion 10a may be configured to be inserted into the external auditory canal of the subject. That is, the first wearing portion 10a may be configured such that the sound output hole 12 is inserted into the external auditory canal of the subject when the biosensor 1 is worn on the ear of the subject. For example, the first wearing portion 10a may be longer than the state illustrated in FIG. 5. Further, in this case, the external auditory canal of the subject may be configured to be closed by the sound output hole 12. In this manner, the subject can listen to the sound or the voice output from the sound output hole 12, and further, can block the sound of the surrounding environment. Therefore, the subject can increase a sense of immersion while measuring the biological information by using the biosensor 1. Further, the subject can hear the sound or the voice output from the sound output hole 12 more clearly while measuring the biological information by using the biosensor 1.

Next, a mechanism of wearing the biosensor 1 on the ear of the subject will be described.

It is required, first, that the main body 10 of the biosensor 1 can be worn on the ear of the subject, and after that, it is required that the helix of the subject is sandwiched with a moderate force. In order to realize such configuration, in the biosensor 1 according to an embodiment, at least a part of the main body 10 may be configured to have elasticity.

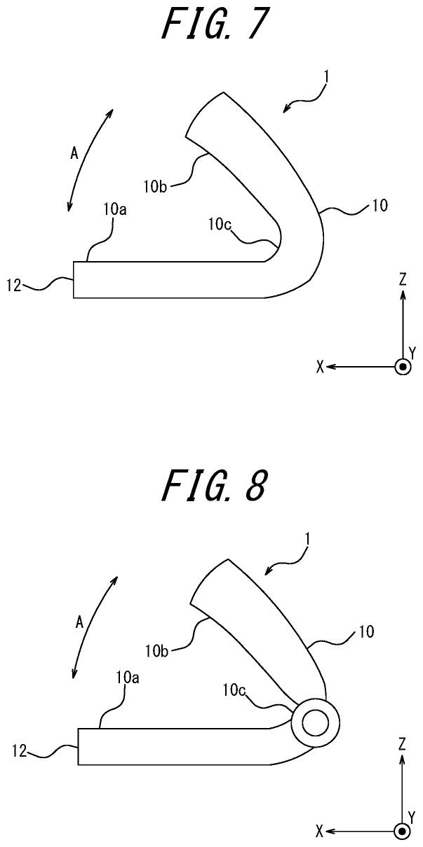

For example, as illustrated in FIG. 7, the connecting portion 10c of the main body 10 may have elasticity. This elasticity may give an elastic force to allow the first wearing portion 10a and the second wearing portion 10b to close to each other. In this case, the subject or the inspector can spread the main body 10 as a whole (in the direction of arrow A in FIG. 7) by opening the first wearing portion 10a and the second wearing portion 10b to each other. FIG. 7 illustrates a state where the first wearing portion 10a and the second wearing portion 10 are opened to each other in the main body 10 of the biosensor 1. In this manner, in the state in which the first wearing portion 10a and the second wearing portion 10b are opened to each other, the subject or the inspector can easily position the first wearing portion 10a and the second wearing portion 10b with respect to the helix of the subject. When the first wearing portion 10a and the second wearing portion 10b are positioned with respect to the helix of the subject, the subject or the inspector can gradually weaken the force to open the first wearing portion 10a and the second wearing portion 10b. Then, when the subject or the inspector releases the force to open the first wearing portion 10a and the second wearing portion 10b, the first wearing portion 10a and the second wearing portion 10b will sandwich the helix of the subject with an appropriate force with the elastic force of the connecting portion 10c.

In this manner, in the biosensor 1 according to an embodiment, the connecting portion 10c may connect the first wearing portion 10a and the second wearing portion 10b so that they are displaceable to each other. Further, in the biosensor 1 according to an embodiment, the main body 10 may be configured such that the helix of the subject is sandwiched by the elasticity of at least one of the first wearing portion 10a, the second wearing portion 10b and the connecting portion 10c.

Further, as illustrated in FIG. 8, the connecting portion 10c may have a rotatable mechanism. The connecting portion 10c may give a force in the direction in which the first wearing portion 10a and the second wearing portion 10b are close to each other. Although the rotatable mechanism of the connecting portion 10c illustrated in FIG. 8 can be rotated by applying a force equal to or greater than a predetermine value, it may be configured to be remained fixed and not to rotate even if a force less than the predetermined value is applied. In this case, the subject or the inspector can spread the main body 10 as a whole (in the direction of the arrow A in FIG. 8) by applying a force equal to or greater than the predetermined value to the first wearing portion 10a and the second wearing portion 10b to rotate the connecting portion 10c. FIG. 8 illustrates a state of the main body 10 of the biosensor 1 in which the first wearing portion 10a and the second wearing portion 10b are opened to each other. In this manner, in the state in which the first wearing portion 10a and the second wearing portion 10b are opened to each other, the subject or the inspector can easily position the first wearing portion 10a and the second wearing portion 10b with respect to the helix of the subject. When positioning the first wearing portion 10a and the second wearing portion 10b with respect to the helix of the subject, the subject or the inspector can close the main body 10 as a whole by applying a force that is equal to or greater than the predetermined value again to the first wearing portion 10a and the second wearing portion 10b to rotate the connecting portion 10c. Then, when the subject or the inspector releases a force to close the first wearing portion 10a and the second wearing portion 10b to each other, the first wearing portion 10a and the second wearing portion 10b will sandwich the helix of the subject with an appropriate force due to the elastic force of the connecting portion 10c.

Next, the measurement unit of the biosensor 1 will be described.

The biosensor 1 can measure at least one of the percutaneous oxygen saturation (SpO₂) and the blood flow amount of the subject. Thus, the biosensor 1 has a measurement unit that measures at least one of the percutaneous oxygen saturation (SpO₂) and the blood flow amount of the subject.

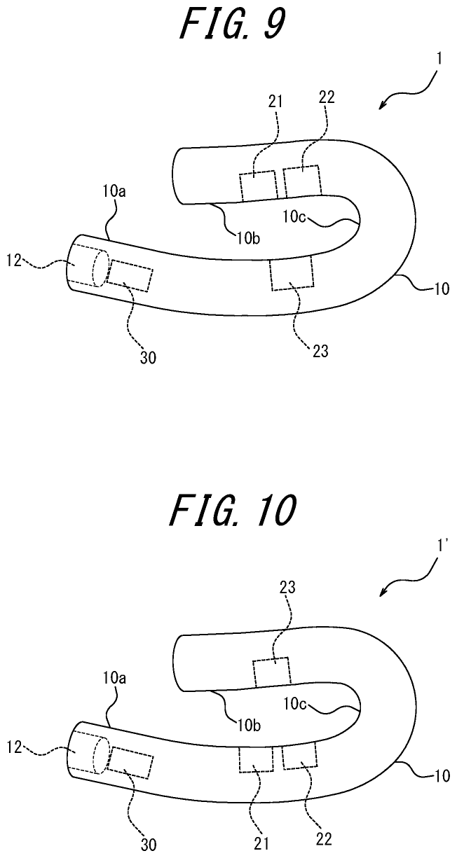

FIG. 9 is a diagram illustrating a configuration of the measurement unit of the biosensor 1 according to an embodiment.

As illustrated in FIG. 9, the biosensor 1 according to an embodiment may have a first light source 21, a second light source 22 and a light receiver 23. FIG. 9 illustrates a state in which all of the first light source 21, the second light source 22 and the light receiver 23 are built in the main body 10. Thus, the first light source 21, the second light source 22 and the light receiver 23 are indicated by the dashed lines in FIG. 9. In the following, the first light source 21 and the second light source 22 are described also as light emitters (21, 22). Further, the first light source 21, the second light source 22 and the light receiver 23 are described as a measurement unit 20 as appropriate.

The first light source 21 and the second light source 22 may emit, as measurement light, laser light having a wavelength at which a predetermined component contained in blood can be detected. The first light source 21 and the second light source 22 may respectively be configured as a Laser Diode (LD), for example. As a laser light source used by this embodiment, a Vertical Cavity Surface Emitting Laser (VCSEL) may be used, for example. However, other lasers such as a Distributed Feedback (DFB) laser and Fabry-Perot (FP) laser may be used. In an embodiment, at least one of the first light source 21 and the second light source 22 may be configured as a Light Emitting Diode (LED).

The first light source 21 and the second light source 22 emit laser light of different wavelengths. The first light source 21 emits laser light of a first wavelength (hereinafter referred to as “first laser light”). The first wavelength is a wavelength having a large difference between the absorbance of hemoglobin bound to oxygen (hereinafter also referred to as “oxygenated hemoglobin”) and the absorbance of hemoglobin not bounded to oxygen (hereinafter also referred to as “reduced hemoglobin”). The first wavelength is a wavelength of 600 nm to 700 nm, for example, and the first laser light is what is called red light. This embodiment will be described below on the assumption that the first wavelength is 660 nm. The second light source 22 emits laser light of a second wavelength (hereinafter also referred to as “second laser light”). The second wavelength is different from the first wavelength. The second wavelength has a smaller difference between the absorbance of the oxygenated hemoglobin and the absorbance of the reduced hemoglobin than the first wavelength. The second wavelength is a wavelength of 800 nm to 1000 nm, for example, and the second laser light is what is called near infrared light. In this embodiment, description will be given below on the assumption that the second wavelength is 850 nm.

The light receiver 23 receives, as a biological measurement output, the scattered light (detection light) irradiated to the measured part and scattered from the measured part. The light receiver 23 may be configured by a Photo Diode (PD), for example. In an embodiment, the light receiver 23 may be configured by a PD that can detect wavelengths of both red light and near-infrared light. The biosensor 1 may transmit a photoelectric conversion signal received at the light receiver 23 to an external device via the cable 14, for example.

As illustrated in FIG. 9, the light emitting faces of the first light source 21 and the second light source 22 are disposed to be exposed from the second wearing portion 10b. In this manner, the first light source 21 and the second light source 22 can appropriately irradiate light to the helix of the subject. Further, as illustrated in FIG. 9, the light emitting face of the light receiver 32 is disposed to be exposed from the first wearing portion 10a. In this manner, the light receiver 32 can appropriately receive the light that is irradiated from at least one of the first light source 21 and the second light source 22 and is transmitted through the helix of the subject.

In the main body 10 of the biosensor 1 illustrated in FIG. 9, the first light source 21 and the second light source 22, that is, light emitters (21, 22), are disposed on the second wearing portion 10b side. Further, in the main body 10 of the biosensor 1 illustrated in FIG. 9, the light receiver 23 is disposed on the first wearing portion 10a side. With the above described disposition, the measurement unit 20 of the biosensor 1 constitutes a transmission type measurement unit. That is, in the measurement unit 20 of the biosensor 1, at least a part of the light emitted from the light emitters (21, 22) transmits the helix of the subject and is received by the light receiver 23. Therefore, the measurement unit 20 of the biosensor 1 can measure at least one of the percutaneous oxygen saturation (SpO₂) and the blood flow amount of the subject while being worn on the helix of the subject.

Further, as illustrated in FIG. 9, a sound output interface 30 may be built in the sound output hole 12 formed at the end of the first wearing portion 10a. Here, the sound output interface 30 may be configured by any member that can transmit sound or voice through at least one of air vibration and bond conduction. For example, the sound output interface 30 may be configured by various members such as a dynamic receiver, a bone conduction receiver, or a smart sonic receiver.

The sound output interface 30 makes the subject to listen to any music or announcement of instructions while the subject wears the biosensor 1 on his/her helix and measures the biological information. Further, the sound output interface 30 may output the information based on the biological information measured by the biosensor 1 by sound or voice. For example, the sound output interface 30 may make the subject to listen to the measurement result of the biological information by the biosensor 1 as a voice announcement. Further, for example, the sound output interface 30 may allow the subject to listen to the measurement results of the biological information by the biosensor 1 by predetermined warning sound or music to call attention.

In this manner, in the biosensor 1 according to an embodiment, the first wearing portion 10a may have the sound output interface 30 that outputs sound from the end (sound output hole 12) of the first wearing portion 10a. In this case, the sound output interface 30 may transmit the sound by at least one of air vibration and bone conduction.

FIG. 10 is a diagram illustrating a configuration of the measurement unit of the biosensor according to a variation of the biosensor 1 illustrated in FIG. 9.

As illustrated in FIG. 10, in a biosensor 1′ according to an embodiment, the positions of the light emitters (21, 22) and the light receiver 23 are reversed in the biosensor 1 illustrated in FIG. 9. That is, in the main body 10 of the biosensor 1′ illustrated in FIG. 10, the first light source 21 and the second light source 22, that is, the light emitters (21, 22), are disposed on the first wearing portion 10a side. Further, in the main body 10 of the biosensor 1′ illustrated in FIG. 10, the light receiver 23 is disposed on the second wearing portion 10b side. The other configurations may be the same as those of the biosensor 1 illustrated in FIG. 9. Therefore, the measurement unit 20 of the biosensor 1′ can also measure at least one of the percutaneous oxygen saturation (SpO₂) and the blood flow amount of the subject while being worn on the helix of the subject. In the biosensor 1′ illustrated in FIG. 10, the light receiver 23 is disposed on the front side of the outer ear (auricle). Thus, in the biosensor 1′ illustrated in FIG. 10, the light receiver 23 is less likely to receive light such as sunlight, that is, light other than the light emitted from the light emitters (21, 22). Therefore, the biosensor 1′ illustrated in FIG. 10 can measure with less noise.

As the biosensors 1 and 1′ illustrated in FIGS. 9 and 10, the light emitters (21, 22) are disposed on either one of the first wearing portion 10a and the second wearing portion 10b, and the light receiver 23 may be disposed on the other.

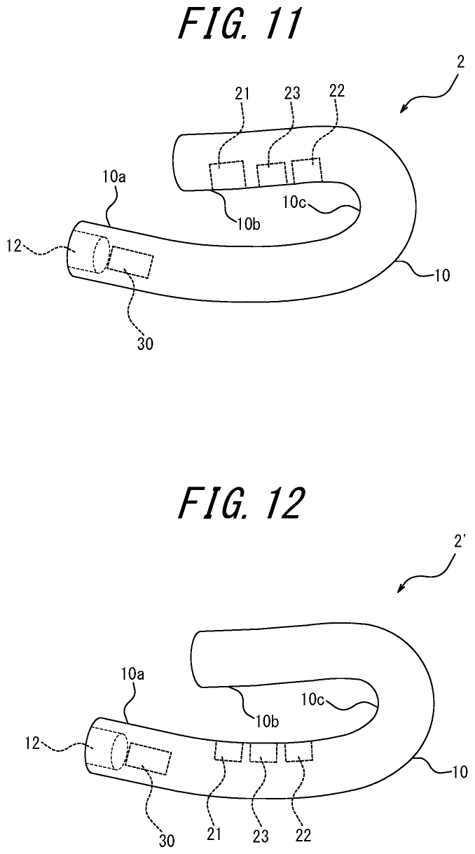

FIG. 11 is a diagram illustrating a configuration of the measurement unit of the biosensor according to another variation of the biosensor 1 illustrated in FIG. 9.

In the main body 10 of the biosensor 2 illustrated in FIG. 11, the first light source 21 and the second light source 22, that is, the light emitters (21, 22), and the light receiver 23 are disposed on the second wearing portion 10b side. The other configurations may be the same as the biosensor 1 or 1′ illustrated in FIG. 9 or FIG. 10. In this manner, the measurement unit 20 of the biosensor 2 constitutes a reflective measurement unit. That is, in the measurement unit 20 of the biosensor 2, at least a part of the light irradiated from the light emitters (21, 22) is reflected by the helix of the subject and is received by the light receiver 23. Therefore, the measurement unit 20 of the biosensor 2 can measure at least one of the percutaneous oxygen saturation (SpO₂) and the blood flow amount of the subject while being worn on the helix of the subject. In this case, the measurement unit 20 may simultaneously measure the SpO₂ and the blood flow amount.

FIG. 12 is a diagram illustrating a configuration of the measurement unit of the biosensor according to a variation of the biosensor 2 illustrated in FIG. 11.

As illustrated in FIG. 12, in a biosensor 2′ according to an embodiment, the position of the measurement unit 20 is reversed in the biosensor 2 illustrated in FIG. 11. That is, in the main body 10 of the biosensor 2′ illustrated in FIG. 12, the first light source 21 and the second light source 22, that is, the light emitters (21, 22), and the light receiver are disposed not on the second wearing portion 10b side, but on the first wearing portion 10a side. The other configurations may be the same as those of the biosensor 2 illustrated in FIG. 11. Therefore, the measurement unit 20 of the biosensor 2′ can also measure at least one of the percutaneous oxygen saturation (SpO₂) and the blood flow amount of the subject while worn on the helix of the subject. In the biosensor 2′ illustrated in FIG. 12, the light receiver 23 is disposed on the front side of the outer ear (auricle). Thus, in the biosensor 2′ illustrated in FIG. 12, the light receiver 23 is less likely to receive light such as sunlight, that is, light other than the light emitted from the light emitters (21, 22). Therefore, the biosensor 2′ illustrated in FIG. 12 can measure with less noise.

As in the biosensors 2 and 2′ illustrated in FIGS. 11 and 12, both the light emitters (21, 22) and the light receiver 23 may be disposed on one of the first wearing portion 10a and the second wearing portion 10b.

In this manner, the biosensor 1 according to an embodiment has the measurement unit 20. Further, in the biosensor 1 according to an embodiment, the measurement unit 20 measures at least one of the percutaneous oxygen saturation (SpO₂) and the blood flow amount of the subject. Further, in the biosensor 1 according to an embodiment, the measurement unit 20 may have the light emitters (21, 22) and the light receiver 23. Further, the light emitters (21, 22) may have the first light source 21 and the second light source 22. Thus, according to the biosensor 1 of an embodiment, the biological information of the subject can be stably measured when the biological information of the subject is measured. Therefore, according to the biosensor of an embodiment, the convenience can be improved.

Further, as illustrated in FIG. 9-FIG. 12, in the biosensors 1, 1′, 2 and 2′ according to an embodiment, the measurement unit 20 may be disposed at least one of the first wearing portion 10a and the second wearing portion 10b.

Next, the measurement apparatus, which is an external device connected to the biosensor 1, will be described.

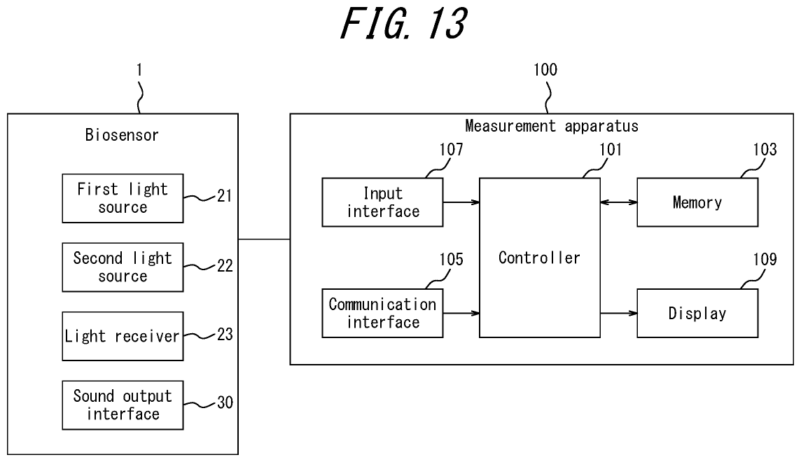

FIG. 13 is a functional block diagram illustrating a schematic configuration of the biosensor 1 illustrated in FIG. 9 and the measurement apparatus 100 connected to the biosensor 1.

As illustrated in FIG. 13, the biosensor 1 has the first light source 21, the second light source 22, the light receiver 23 and the sound output interface 30. Since these functions have already been described, detailed description will be omitted.

As illustrated in FIG. 13, the biosensor 1 may be connected to the measurement apparatus 100, which is an external device. In this case, the biosensor 1 may be connected to the measurement apparatus 100 via the cable 14. The biosensor 1 may integrally have all of or at least a part of the measurement apparatus 100 illustrated in FIG. 13 in the biosensor 1.

As illustrated in FIG. 13, the measurement apparatus 100 may have a controller 101, a memory 103, a communication interface 105, an input interface 107 and a display 109. The measurement apparatus 100 may be any external device connectable to the biosensor 1. For example, the measurement apparatus 100 may be a terminal dedicated to be connected to the biosensor 1. Further, the measurement apparatus 100 may be any existing electronic device such as, for example, a smart phone, a tablet terminal, a notebook computer, or a general-purpose computer. In this case, these electronic devices may be activated with application software for measuring the biological information of the subject by the biosensor 1 and estimating the state of the subject on the basis of the biological information measured. The biosensor 1 may be powered by an internal battery or an external power source.

The controller 101 entirely controls and manages at least one of the biosensor 1 and the measurement apparatus 100, including each functional block of at least one of the biosensor 1 and the measurement apparatus 100. The controller 101 may be configured by including at least one processor. The controller 101 may be configured by including at least one processor such as a Central Processing Unit (CPU) configured to execute a program that defines a control procedure, and realizes its function. Such a program may be stored, for example, in the memory 103 or an external storage medium connected to the measurement apparatus 100.

According to various embodiments, at least one processor may be implemented as a single integrated circuit (IC), or a plurality of communicably connected integrated circuits IC and/or discrete circuits. At least one processor can be configured according to various known technologies.

In an embodiment, the processor includes one or more circuits or units configured to execute one or more data computing procedures or processes by executing instructions stored in an associated memory, for example. In other embodiments, the processor may be firmware (e.g., a discrete logic component) configured to execute one or more data computing procedures or processes.

According to various embodiments, the processor may include one or more processors, controllers, microprocessors, microcontrollers, application specific integrated circuits (ASICs), digital signal processors, programmable logic devices, field programmable gate arrays, or any combination of these devices or configurations or any combination of other known devices or configurations, and may perform the functions of the controller 101 described below.

The controller 101 controls, for example, measurement processing of the biological information. For example, the controller 101 controls measurement processing of SpO₂ of the subject by the biosensor 1. The controller 101 may estimate the state of the subject on the basis of the measured information. In this embodiment, for example, the controller 101 may estimate the possibility that the subject develops altitude sickness (also called altitude impairment) on the basis of SpO₂ of the subject measured. The subject is more likely to develop altitude sickness when SpO₂ decreases.

The controller 101 may notify the measured biological information and/or the estimated possibility that the subject develops altitude sickness to the subject via the sound output interface 30 by controlling the sound output interface 30. Further, the controller 101 may notify such information to the subject via the display 109 by controlling the display 109. In this manner, the subject can learn the notified information. For example, when receiving a notification that the possibility that the subject develops altitude sickness is high, the subject can take a measure to prevent altitude sickness beforehand.

The memory 103 can be configured by a semiconductor memory, a magnetic memory, or the like. The memory 103 stores various kinds of information and a program for operating the measurement apparatus 100. The memory 103 may also function as a working memory. The memory 103 may store, for example, the body temperature and SpO₂ of the subject calculated by the controller 101, as history information. The memory 103 may store the information about the possibility that the subject develops altitude sickness estimated by the controller 101.

In an embodiment, the memory 103 may store the information of the sound output by the sound output interface 160. Here, the information of the sound stored in the memory 103 may be a voice file of any type such as MP3 (MPEG-1 Audio Layer-3) file or WAV file, for example. In an embodiment, the memory 103 may store various kinds of sound information according to the situation of the subject who uses the measurement apparatus 100.

The communication interface 105 transmits/receives various kinds of data to/from an external device such as the biosensor 1 or an external server through wired or wireless communication. The communication interface 304 can transmit/receive information by using network of wired, wireless or combination of wired and wireless. The communication interface 105 can communicate by, for example, Bluetooth®, infrared rays, NFC, wireless LAN, wired LAN or any other communication media or any combination thereof.

The communication interface 105 may communicate with an external device that stores the biological information of the subject to control the health state. In this case, the communication interface 105 may transmit the measurement results by the biosensor 1 and/or the health state estimated by the measurement apparatus 100 to the external device. Further, when the measurement apparatus 100 is connected to the biosensor 1 via the cable 14, the communication interface 105 may be an interface connecting the cable 14, for example.

The input interface 107 may be configured by including physical keys such as a keyboard and the like or by including a touch panel. The input interface 107 is not limited thereto and may be configured by including various input devices. In an embodiment, the measurement apparatus 100 may start control of measuring the biological information of the subject by the biosensor 1 on the basis of operation input by an operator to the input interface 107.

The display 109 notifies the information by characters, images, and the like. The display 109 may be a display device such as a Liquid Crystal Display (LCD), an Organic Electro-Luminescence Display (OELD:),an Inorganic Electro-Luminescence Display (IELD), and the like. In an embodiment, the display 109 may display the biological information of the subject measured by the biosensor 1 and/or various kinds of information based on the biological information. In this manner, the subject or the inspector can recognize the biological information of the subject and/or various kinds of information based on the biological information. In an embodiment, the display 109 may display the information output from the sound output interface 30 as the information such as characters or images.

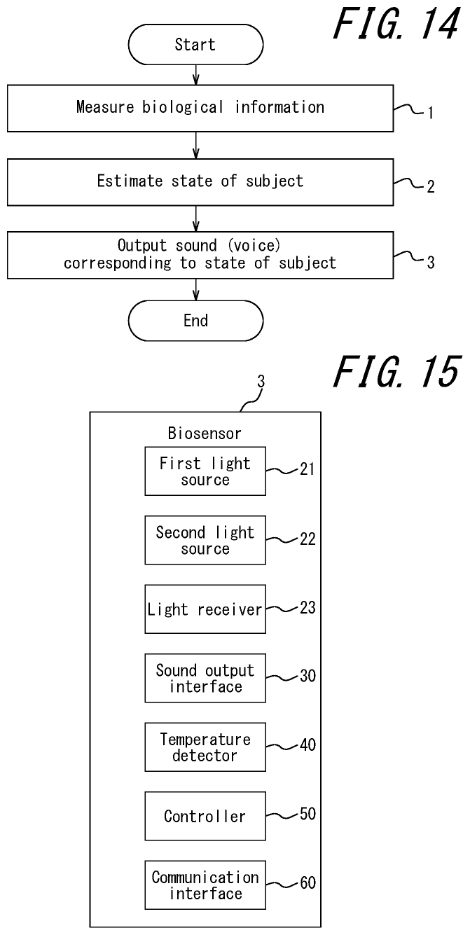

FIG. 14 is a flowchart illustrating an operation executed by the measurement apparatus 100. The measurement apparatus 100 may start the operation illustrated in FIG. 14 when the subject wears the biosensor 1 connected to the measurement apparatus 100 on his/her ear and performs input operation to execute measurement processing to the input interface 107.

When the processing illustrated in FIG. 14 is started, the controller 101 of the measurement apparatus 100 measures the biological information (step S1). More specifically, the measurement apparatus 100 measures the biological information of the helix of the subject by the measurement unit 20 of the biosensor 1. Here, the biological information measured by the measurement unit 20 of the biosensor 1 may be SpO₂ of the subject, for example. The information on the SpO₂ measured by the measurement unit 20 of the biosensor 1 is transmitted to the controller 101 of the measurement apparatus 100. The measurement apparatus 100 according to an embodiment may, in step S1, store the results of measurement by the measurement unit 20 of the biosensor 1 in the memory 103, for example.

The controller 101 of the measurement apparatus 100 estimates the state of the subject on the basis of the measured biological information (step S2). More specifically, the controller 101 may estimate the possibility that the subject develops altitude sickness on the basis of SpO₂ of the subject, for example.

In step S2, the controller 101 of the measurement apparatus 100 according to an embodiment may estimate the state of the subject on the basis of the information measured by the measurement unit 20 of the biosensor 1. For example, the controller 101 may estimate that the possibility that the subject develops altitude sickness is high when a predetermined condition that all measured values of the SpO₂ of the subject exceed a predetermined threshold is met. Further, for example, when the SpO₂ of the subject is within a predetermined range, the controller 101 may estimate that the subject is in a predetermined health state.

The controller 101 notifies the information to the subject via the sound output interface 30 by transmitting a control signal to the sound output interface 30 (step S3). For example, the controller 101 may notify the information by letting the subject to hear a predetermined sound or voice.

The measurement apparatus 100 may repeatedly execute from step S1 to S3 periodically, irregularly or continuously. In this manner, the measurement apparatus 100 can continuously obtain the biological information of the subject and the history of the state of the subject.

The measurement apparatus 100 may notify the information by the means other than the sound output interface 30 in step S3. For example, the measurement apparatus 100 may display the information on the display 109 to notify the information. Further, the measurement apparatus 100 may notify the information by any other means that can be recognized by the subject.

In this manner, the biosensor 1 according to an embodiment may output, as at least one of sound and voice, the information based on at least one of the percutaneous oxygen saturation (SpO₂) and the blood flow amount of the subject measured by the measurement unit 20 from the sound output interface 30.

Next, the biosensor according to another embodiment will be described.

The biosensor 1 illustrated in FIG. 13 was described on the assumption that it has a function of measuring the biological information of the subject and a function of outputting sound or voice to the ear of the subject. That is, the biosensor 1 illustrated in FIG. 13 was described as a biosensor that has no function of processing the biological information, and the measurement apparatus 100 connected to the biosensor 1 processes the biological information.

However, the biosensor according to another embodiment may have a function of processing the biological information by itself, for example. Such an embodiment will be described below.

FIG. 15 is a functional block diagram illustrating a schematic configuration of a biosensor according to another embodiment. As illustrated in FIG. 15, the biosensor 3 according to another embodiment includes a first light source 21, a second light source 22, a light receiver 23, a sound output interface 30, a temperature detector 40, a controller 50 and a communication interface 60. Since the first light source 21, the second light source 22, the light receiver 23 and the sound output interface 30 have already been described, a more detailed description will be omitted.

As illustrated in FIG. 15, the biosensor 3 may include the temperature detector 40. The temperature detector 40 may be any temperature sensor capable of detecting the temperature of a contact portion, such as a thermistor, for example. The temperature detector 40 may be disposed at any position of the main body 10 where the body temperature of the subject can be detected. For example, the temperature detector 40 may be disposed near the measurement unit 20 on at least one of the first wearing portion 10a and the second wearing portion 10b. In this manner, the temperature detector 40 can detect the temperature of the helix of the subject, that is, the body temperature of the subject.

The biosensor 3 may output the information on the body temperature of the subject detected by the temperature detector 40 as sound or voice from the sound output interface 30, for example. Further, the biosensor 3 may consider the body temperature of the subject detected by the temperature detector 40 when detecting the state of the subject.

The controller 50 may be a function part that executes functions similar to those of the controller 101 of the measurement apparatus 100 illustrated in FIG. 13. The biosensor 3 may have a built-in controller 50 in any portion of the main body 10. For example, the second wearing portion 10b of the main body 10 may be formed larger than the first wearing portion 10a and have a built-in small controller 50.

In this manner, the biosensor 3 according to an embodiment may have the controller 50 that performs a predetermined processing to the information on at least one of the percutaneous oxygen saturation (SpO₂) and the blood flow amount of the subject measured by the measurement unit 20. Since the biosensor 3 has the controller 50, it can estimate the state of the subject on the basis of the biological information of the subject measured by the measurement unit 20 without being connected to the external device such as the measurement apparatus 100 illustrated in FIG. 13. That is, the biosensor 3 can perform complete functions independently without being connected to the external device. Further, the biosensor 3 according to an embodiment may output the information based on at least one of the percutaneous oxygen saturation (SpO₂) and the blood flow amount of the subject measured by the measurement unit 20 as at least one of sound and voice from the sound output interface 30.

The communication interface 60 may be a function part that executes functions similar to those of the communication interface 105 of the measurement apparatus 100 illustrated in FIG. 13. The biosensor 3 according to an embodiment may transmit the results measured by the biosensor 1 and/or the health state estimated by the controller 50 to the external device such as an external server, for example. In this manner, the biosensor 3 according to an embodiment may have the communication interface 60 connected wired or wirelessly to the external terminal device, Further, when the biosensor 1 is connected to the external device via the cable 14, the communication interface 60 may be an interface that connects the cable 14, for example.

As described above, according to the biosensor of an embodiment, when measuring the biological information of a subject, the biological information can be measured stably while reducing physical and mental load on the subject. Thus, according to the biosensor of an embodiment, the convenience can be improved.

When worn on the ear of the subject, the biosensor according to an embodiment can measure with the light intensity that is almost the same as the case where the biological information of the subject is measured by irradiating the earlobe with light. Therefore, according to the biosensor of an embodiment, measurement can be made with less light intensity compared to the case where measurement is made by irradiating the finger of the subject with light, for example. Thus, according to the biosensor of an embodiment, low power consumption can be realized compared to the conventional general measuring instrument.

Further, the biosensor according to an embodiment enables measurement of the biological information in the natural state of the subject without forcing the subject to take an uncomfortable posture and without giving a sense of discomfort to the subject. Therefore, the biosensor according to an embodiment can minimize the load on the subject such as a feeling of fatigue. Further, since the biosensor according to an embodiment is configured to be wearable, it can be used even when the subject is moving, such as during exercise. Further, according to the biosensor of an embodiment, since the biosensor is stably positioned to the helix of the subject, it can stably measure the biological information of the subject.

Although the present disclosure has been described on the basis of the drawings and the examples, it is to be noted that various changes and modifications may be made easily by those who are ordinarily skilled in the art on the basis of the present disclosure. Accordingly, it is to be noted that such changes and modifications are included in the scope of the present disclosure. For example, functions and the like included in each component or each step can be rearranged without logical inconsistency, and a plurality of components or steps can be combined into one or divided. Although the embodiment according to the present disclosure has been described mainly on the apparatus, the embodiment according to the present disclosure can also be realized as a method including steps executed by each component of the apparatus. The embodiments according to the present disclosure can also be realized as a method and a program executed by a processor included in the apparatus, or a storage medium on which a program is recorded. It should be understood that the scope of the present disclosure includes these as well. Although the present disclosure has been described on the basis of the drawings and the examples, it is to be noted that various changes and modifications may be made easily by those who are ordinarily skilled in the art on the basis of the present disclosure. Accordingly, it is to be noted that such changes and modifications are included in the scope of the present disclosure. For example, functions and the like included in each function part can be rearranged without logical inconsistency, and a plurality of function parts can be combined into one or divided. Each embodiment according to the above described disclosure is not limited to being faithfully implemented in accordance with the above described each embodiment, and may be implemented by appropriately combining each feature or omitting a part thereof. That is, those who are ordinarily skilled in the art can make various changes and modifications to the contents of the present disclosure on the basis of the present disclosure. Therefore, such changes and modifications are included in the scope of the present disclosure. For example, in each embodiment, each function, each means, each step and the like may be added to another embodiment without logical inconsistency, or replaced with each function, each means, each step and the like of another embodiment. Further, in each embodiment, a plurality of functions, means or steps can be combined into one or divided. Moreover, each embodiment according to the above described disclosure is not limited to being faithfully implemented in accordance with the above described each embodiment, and may be implemented by appropriately combining each feature or omitting a part thereof.

### REFERENCE SIGNS LIST

1, 1′, 2, 2′ Biosensor

10 Main body

10a First wearing portion

10b Second wearing portion

10c Connecting portion

12 Sound output hole

14 Cable

16 Cable connecting unit

21 First light source

22 Second light source

23 Light receiver

30 Sound output interface

40 Temperature detector

50 Controller

60 Communication interface

100 Measurement apparatus

101 Controller

103 Memory

105 Communication interface

107 Input interface

109 Display

1. A biosensor, comprising:
a main body configured to sandwich a helix of a subject by a first wearing portion and a second wearing portion; and
a measurement unit configured to measure at least one of percutaneous oxygen saturation (SpO₂) and blood flow amount of the subject.

2. The biosensor according to claim 1, wherein the main body comprises a connecting portion configured to connect the first wearing portion and the second wearing portion.

3. The biosensor according to claim 2, wherein the connecting portion connects the first wearing portion and the second wearing portion displaceable to each other.

4. The biosensor according to claim 2, wherein the main body is configured to sandwich the helix of the subject by elasticity of at least one of the first wearing portion, the second wearing portion and the connecting portion.

5. The biosensor according to claim 1, wherein the measurement unit is disposed on at least one of the first wearing portion and the second wearing portion.

6. The biosensor according to claim 1, wherein the measurement unit includes a light emitter and a light receiver.

7. The biosensor according to claim 6, wherein the light emitter is disposed on one of the first wearing portion and the second wearing portion, and the light receiver is disposed on the other one of the first wearing portion and the second wearing portion.

8. The biosensor according to claim 6, wherein both the light emitter and the light receiver are disposed on one of the first wearing portion and the second wearing portion.

9. The biosensor according to claim 6, wherein the light emitter includes a first light source and a second light source.

10. The biosensor according to claim 1, wherein an end of the first wearing portion is inserted into an external auditory canal of the subject; and
the first wearing portion includes a sound output interface configured to output sound from the end of the first wearing portion.

11. The biosensor according to claim 1, wherein
the end of the first wearing portion is positioned in front of an entry of the external auditory canal of the subject; and
the first wearing portion includes a sound output interface configured to output sound from the end of the first wearing portion.

12. The biosensor according to claim 10, wherein the sound output interface transmits sound by at least one of air vibration and bone conduction.

13. The biosensor according to claim 10, wherein information on the basis of at least one of percutaneous oxygen saturation (SpO₂) and blood flow amount of the subject measured by the measurement unit is output as at least one of sound and voice from the sound output interface.

14. The biosensor according to claim 1, comprising a controller configured to perform predetermined processing to at least one of the information of percutaneous oxygen saturation (SpO₂) and blood flow amount of the subject measured by the measurement unit.

15. The biosensor according to claim 1, comprising a communication interface configured to be connected to an external terminal device wired or wireless.
